(19) United States
(12) Patent Application Publication
(10) Pub. No.: US 2023/0191076 A1
(43) Pub. Date: Jun. 22, 2023

(54) TRAINING DEVICE FOR IMPROVING INSOMNIA

(71) Applicants: Shin-Da LEE, Taichung (TW); Ears on Technology Co., Ltd., Taichung City (TW)

(72) Inventors: Shin-Da LEE, Taichung (TW); Yu-Kuei TENG, Taichung City (TW); Chen-Chao HSU, Taichung City (TW)

(21) Appl. No.: 18/082,880

(22) Filed: Dec. 16, 2022

(30) Foreign Application Priority Data
Dec. 17, 2021 (CN) 202111554747.8

(51) Int. Cl. A61M 21/02; A61N 2/00; A61N 2/02; G16H 40/67; G16H 10/20

(52) U.S. Cl. CPC A61M 21/02; A61N 2/006; A61N 2/002; A61N 2/02; G16H 40/67; G16H 10/20; A61M 2021/0027

(57) ABSTRACT

A training device for improving insomnia is provided, comprising a virtual reality unit and a control unit. The virtual reality unit comprises two earphones and an eye cover connected to the earphones. When the control unit executes the daytime relaxaion-assisted program, the control unit controls the earphones to play a daytime binaural beats with frequency following response. The daytime binaural beats with frequency following response have a first main carrier frequency tone and a first loading frequency tone differing from the first main carrier frequency tone by a first difference frequency rate. When the control unit executes the nighttime anti-insomnia-assisted program, the control unit controls the earphones to play the nighttime difference frequency tone. The nighttime difference frequency tone have a second main carrier frequency tone and a second loading frequency tone differing from the second main carrier frequency tone by a second difference frequency rate.

### BACKGROUND OF THE INVENTION

### Field of the Invention

The present invention relates to a training device and in particular to a training device for improving insomnia

### Description of the Prior Art

Insomnia is a very common disease nowadays. It is known that insufficient melatonin secretion and too much sympathetic nerve excitation can cause insomnia Insufficient sleep can cause harm to human health. It is known that supplemental artificial melatonin has side effects and poor effect. Therefore, how to induce the pineal gland to secrete melatonin is an important key point to solve insomnia. At present, the study on inducing the pineal body to secrete melatonin is still in progress.

### SUMMARY OF THE INVENTION

Accordingly, it is an object of the present invention to provide a training device for improving insomnia that overcomes the problems of the prior art.

Accordingly, a training device for improving insomnia of the present invention comprises a virtual reality unit and a control unit.

The virtual reality unit is configured for being worn by the head of a user, wherein the virtual reality device comprises two earphones, and an eye cover connected to front sides of the earphones and used for displaying a virtual image; the control unit is configured for being electrically connected to the virtual reality unit, with an electronic clock capable of indicating a current time built in, and pre-storing digital information of a daytime binaural beats with frequency following response and a nighttime binaural beats with frequency following response, wherein when the current time is from 8:00 a.m. to 5:00 p.m. the control unit can be used for executing a daytime relaxation-assisted program; when the current time is from 7:00 p.m. to 12:00 p.m. the control unit can be used for executing a nighttime anti-insomnia-assisted program; when the control unit executes the daytime relaxation-assisted program, the control unit controls the earphone to play the daytime binaural beats with frequency following response, and stop playing after continuing for a first predetermined time, wherein the daytime binaural beats with frequency following response has a first main carrier frequency tone and a first loading frequency tone which differs from the first main carrier frequency tone by a first difference frequency rate; the first main carrier frequency tone and the first loading frequency tone are respectively played via the earphones simultaneously; the first difference frequency gradually decreases from a first high frequency to a first low frequency within the first predetermined time; when the control unit executes the nighttime anti-insomnia-assisted program, the control unit controls the earphones to play the nighttime binaural beats with frequency following response, and stops playing after continuing for a second predetermined time; the nighttime binaural beats with frequency following response has a second main carrier frequency tone and a second loading frequency tone which differs from the second main carrier frequency tone by a second difference frequency; the second main carrier frequency tone and the second loading frequency tone are respectively played via the earphones simultaneously; and the second difference frequency rate gradually decreases from a second high frequency which is not higher than the first low frequency to a second low frequency within the second predetermined time.

The efficacy of the present invention lies in that by providing the virtual reality unit and the control unit, the control unit controls the earphones to play the daytime binaural beats with frequency following response and the nighttime binaural beats with frequency following response respectively when the daytime relaxation-assisted program and the nighttime anti-insomnia-assisted program are executed, so that the user can achieve the effect of improving insomnia.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Other features and effects of the invention will be apparent from the embodiments with reference to the drawings, in which:

FIG. 1 is a system block diagram of an embodiment of a training device for improving insomnia according to the present invention;

FIG. 2 is a perspective view of a virtual reality unit, an electronic stimulation unit and an acupoint stimulation unit according to the embodiment;

FIG. 3 is a side view of the virtual reality unit, the electronic stimulation unit and the acupoint stimulation unit according to the embodiment;

FIG. 4 is a rear view of the virtual reality unit, the electronic stimulation unit and the acupoint stimulation unit according to the embodiment;

FIG. 5 is a top view of the virtual reality unit, the electronic stimulation unit and the acupoint stimulation unit according to the embodiment;

FIG. 6 is a side view of the embodiment;

FIG. 7 is a schematic view of a picture displayed when an evaluation program is executed in the embodiment;

FIGS. 8 and 9 is a current sleep quality table;

FIG. 10 is a three-dimensional magnetic resonance imaging vision after a subject listen to a binaural beats with frequency following response in the nighttime;

FIG. 11 is a diagram showing the trend of melatonin change in eight subjects before and after listening to the nightime binaural beats with frequency following response;

FIG. 12 is a diagram showing the trend of melatonin eight subjects before and after listening to normal music; and

FIG. 13 is a statistical chart of ten subjects before and after subjecting to vibration on a vibration bed.

### DETAILED DESCRIPTION OF THE PREFERRED EMBODIMENTS

The patent or application file contains at least one drawing executed in color. Copies of this patent or patent application publication with color drawing(s) will be provided by the Office upon request and payment of the necessary fee.

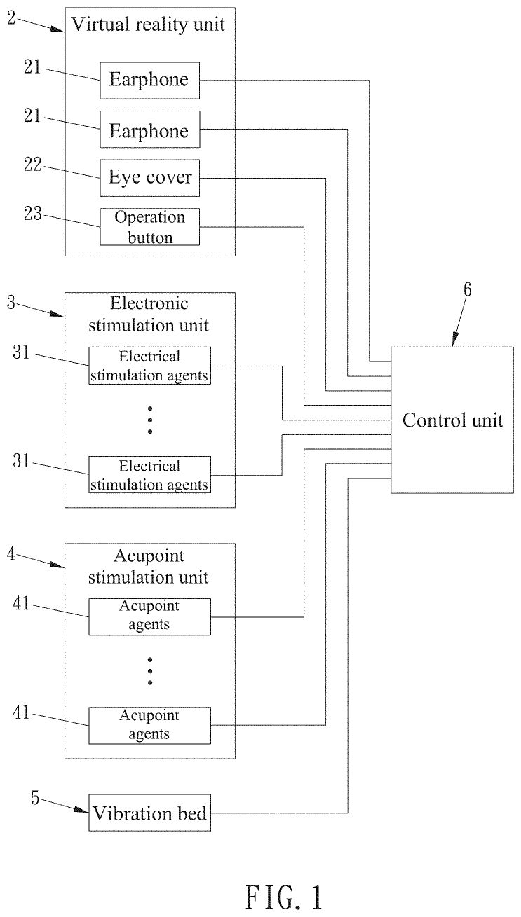

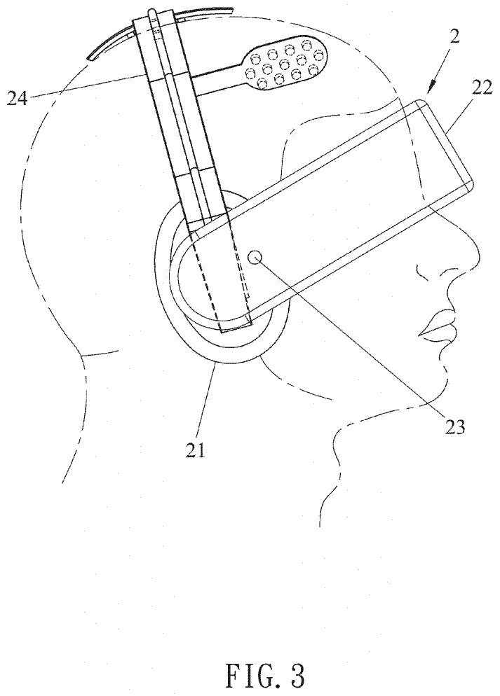

Referring to FIGS. 1, 2 and 3, an embodiment of a training device for improving insomnia of the present invention includes a virtual reality unit 2, an electronic stimulation unit 3, an acupoint stimulation unit 4, a vibration bed 5, and a control unit 6.

The virtual reality unit 2 is configured for being worn by the head of a user, and the virtual reality device 2 includes two earphones 21, an eye cover 22 connected to front sides of the earphones 21 and used for displaying a virtual image, an operation button 23, and a top side frame 24 connected between the earphones 21 and upwardly across the top of the user's head.

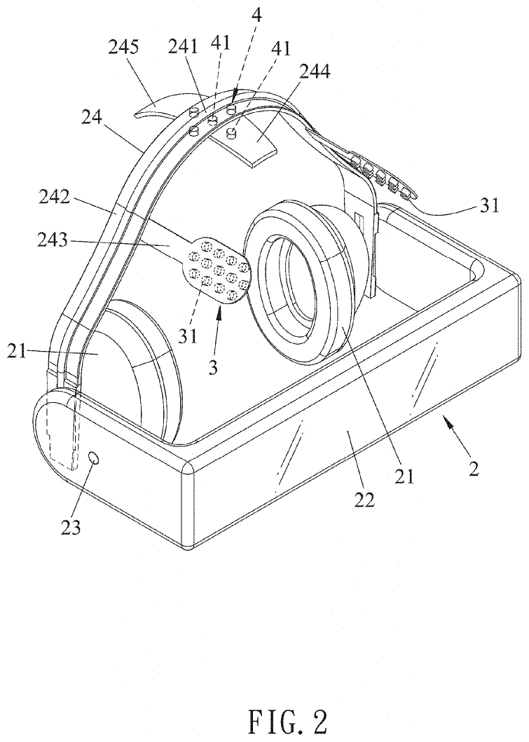

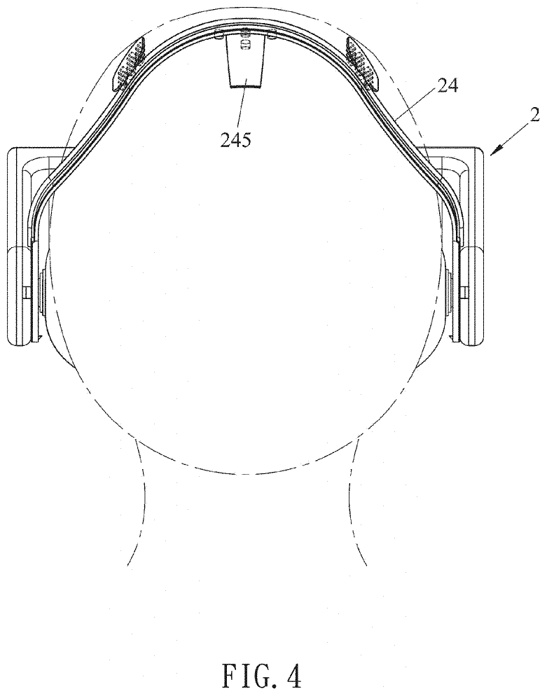

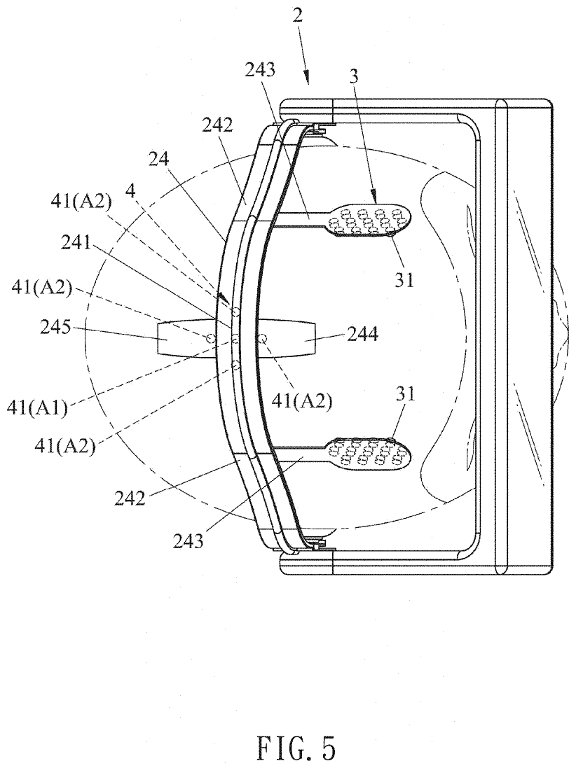

Referring to FIGS. 2, 4 and 5, the top side frame 24 has a top region 241 located at the center between the earphones 21, two side regions 242 respectively connected between the earphones 21 and the top region 241, two connection regions 243 respectively extending forward from the side regions 242, a front extension region 244 extending forwardly from the top region 242, and a rear extension region 245 extending rearwardly from the top region 242.

The electronic stimulation unit 3 includes a plurality of electrical stimulation agents 31 provided at the connection regions 243. The electrical stimulation agents 31 are used for physical stimulation of one of outputting a current to perform transcranial direct current stimulation (tDCS) and outputting an electromagnetic pulse to perform transcranial magnetic stimulation (TMS) on the head of the user. The electrical stimulation agents 31 respectively correspond to an F3 position and an F4 position in international 10-20 system brain wave electrode positions. In the present embodiment, the electrical stimulation agents 31 is used for performing the transcranial direct current stimulation. The magnitude of current is 0.5 mA-2 mA. The current density is 0.03 mA/cm²-0.09 mA/cm². The electromagnetic frequency is 1-20 Hz when the electrical stimulation agents 31 are used for performing the transcranial magnetic stimulation to output electromagnetic pulse.

The acupoint stimulation unit 4 includes a plurality of acupoint agents 41, the acupoint agents being used for outputting physical stimulation of laser light to the user. One of the acupoint agents 41 is adjustably disposed at the center of the top region 241 to correspond to the Baihui acupoint (International acupoint code GV20) of the user, indicated by reference numeral Al in the drawings. Four of the acupoint agents 41 are respectively adjustably disposed at the side regions 242, the front extension region 244, the rear extension region 245, and a position at a distance from the center of the top region 241 by a finger distance to correspond to four Sishencong acupoints (International acupoint code EX-HN1) of the user, respectively, as indicated by reference numeral A2 in the drawings. The finger distance is substantially two to three centimeters.

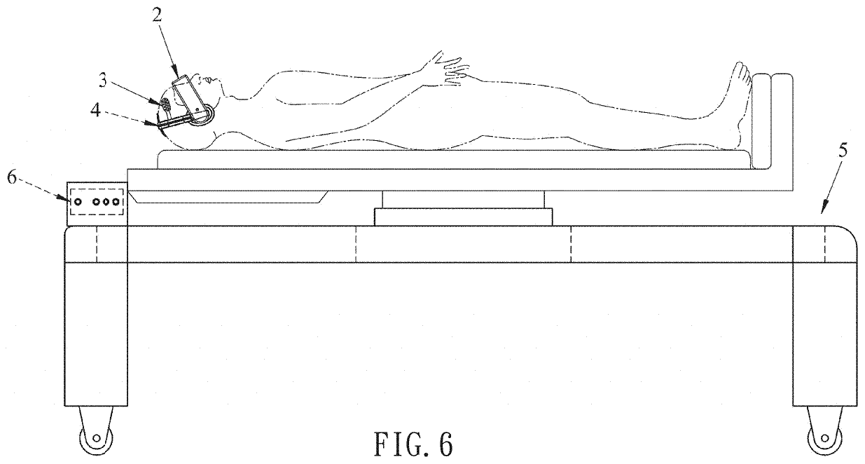

Referring to FIGS. 1 and 6, the vibration bed 5 can be controlled to generate vibrations. The adjustable vibration frequency of the vibration bed 5 is between 6-53 Hz.

The control unit 6 is electrically connected to the earphones 21, the eye cover 22 and the operation button 23 of the virtual reality unit 2. The control unit 6 is electrically connected to the electrical stimulation agents 31, the acupoint agents 41 and the vibration bed 5, and an electronic clock (not shown) indicating a current time is built in. Digital information about a daytime binaural beats with frequency following response and a nighttime binaural beats with frequency following response is pre-stored. When the current time is from 8:00 a.m. to 5:00 p.m. the control unit 6 can be controlled to execute a daytime relaxation-assisted program. When the current time is from 7:00 p.m. to 12:00 p.m., the control unit 6 may be controlled for performing a nighttime anti-insomnia-assisted program. In the present embodiment, the control unit 6 is a microprocessor. The control unit 6 can also be configured for executing a audio-visual education program and an evaluation program.

When the control unit 6 executes the daytime relaxation-assisted program, the control unit 6 controls the headsets 21 to play the daytime binaural beats with frequency following response, and stop playing after continuing for a first predetermined time. The daytime binaural beats with frequency following response has a first main carrier frequency tone and a first loading frequency tone which differs from the first main carrier frequency tone by a first difference frequency rate. The first main carrier frequency tone and the first loading frequency tone are respectively dialed via the headsets 21 simultaneously. The first binaural frequency difference gradually decreases from a first high binaural frequency difference to a first low frequency within the first predetermined time. In this embodiment, the first high binaural frequency difference is 20 Hz, the first low binaural frequency difference is 10 Hz, and the first predetermined time is 30 minutes, thereby enabling the user to improve parasympathetic activity, decrease sympathetic activity, increase voluntary relaxation during the daytime use, and thereby improving the user's insomnia symptoms.

When the control unit 6 executes the daytime relaxation-assisted program, the control unit 6 controls the eye cover 22 to project light with a wavelength of 465-495 nm and an illumination of 2500-3000 lux via the virtual image. In this embodiment, the wavelength and illumination of the light is generated by the hardware of the virtual reality unit 2 in conjunction with the rendering of the virtual image.

If the control unit 6 is requested to perform the daytime relaxation-assisted program when the current time is at a time other than 8:00 a.m. to 5:00 p.m., the control unit 6 does not perform the daytime relaxation-assisted program and displays an unusable warning message on the virtual image to avoid the user's erroneous operation.

When the control unit 6 executes the nighttime anti-insomnia-assisted program, the control unit 6 controls the headsets 21 to play the nighttime binaural beats with frequency following response, and stop playing after continuing for a second predetermined time. The nighttime binaural beats with frequency following response has a second main carrier frequency tone and a second loading frequency tone which differs from the second main carrier frequency tone by a second difference frequency rate. The second main carrier frequency tone and the second loading frequency tone are respectively played via the headsets 21 simultaneously. The second difference frequency rate gradually decreases from a second high frequency which is not higher than the first low frequency to a second low frequency within the second predetermined time. In the present embodiment, the second high frequency is 10 Hz, the second low frequency is 2 Hz, and the second predetermined time is 30 minutes, thereby enabling the user to stimulate the brain and activate the pineal body to produce melatonin by using the nighttime binaural beats with frequency following response during the nighttime use, and then improving the user's insomnia symptoms.

When the control unit 6 executes the nighttime anti-insomnia-assisted program, the control unit 6 controls the eye cover 22 to project light with a wavelength of 630-670 nm and an illumination of less than 30 lux via the virtual image. If the control unit 6 is requested to perform the nighttime anti-insomnia-assisted program when the current time is a time other than from 7:00 p.m. to 12:00 p.m., the control unit 6 does not perform the nighttime anti-insomnia-assisted program, and displays the unusable warning message on the virtual image to avoid the user's erroneous operation.

When the control unit 6 executes the audio-visual education program, the control unit 6 controls the virtual image to display a teaching course and generates a teaching video and audio for teaching insomnia improvement in cooperation with the virtual reality unit 2 and earphones 21.

The contents of the audio-visual education program are, for example, the following contents: first, developing good sleep habits and good daytime and nighttime circadian rhythm can reduce the chances of insomnia. Second, a good sleep environment should be created to facilitate falling asleep and to prevent insomnia. Third, how to eat well and how to eat based on circadian rhythm should be for good sleep quality.

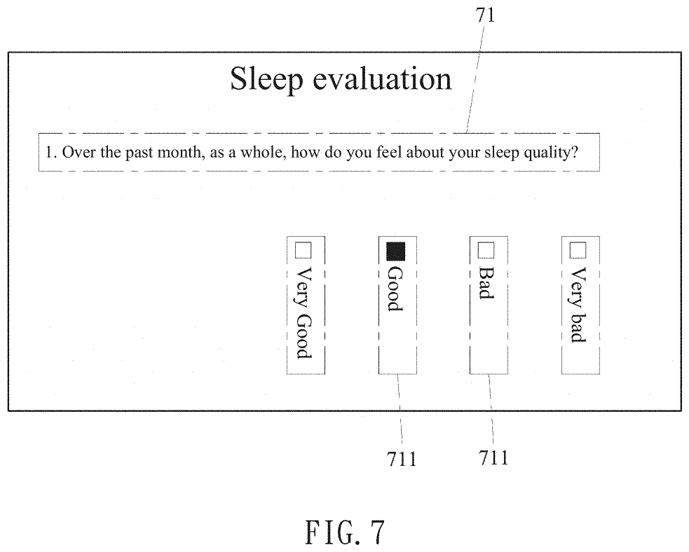

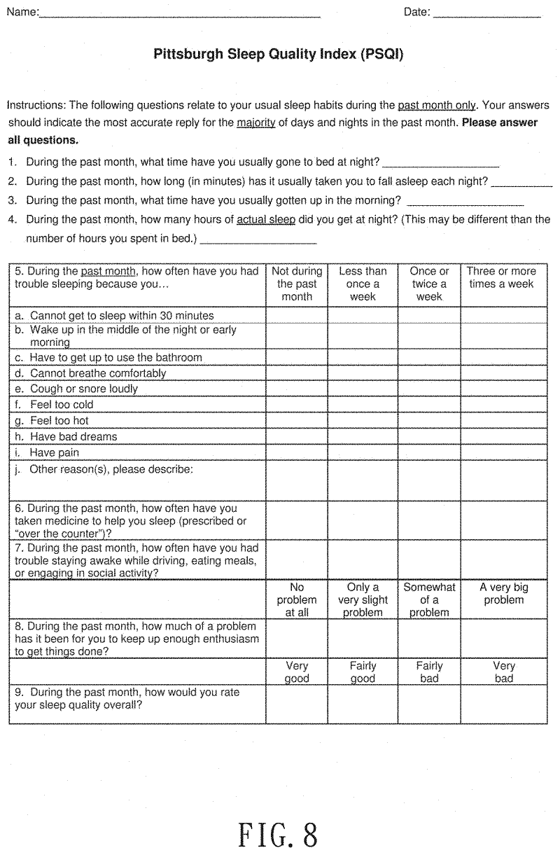

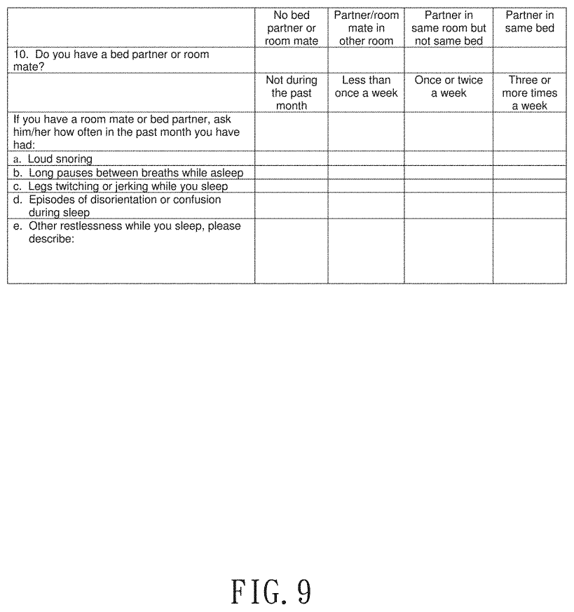

Referring to FIGS. 1, 7 ,8 and 9, when the control unit 6 executes the evaluation program, the control unit 6 controls the virtual image to sequentially display a plurality of insomnia and sleep quality self-evaluation questions 71 for evaluating insomnia, each insomnia and sleep quality self-evaluation question 71 having a plurality of self-evaluation items 711, and the selected self-evaluation items 711 can be changed by changing the direction of the eye cover 22; an insomnia and sleep quality self-score score corresponding to the selected self-evaluation item 711 is generated after confirmation by the operation button 23. Then, after all the insomnia and sleep quality self-assessment questions 71 have been answered, all the insomnia and sleep quality self-assessment scores are summed up to generate an insomnia and sleep quality result score for storage. In the present embodiment, the display module 22 displays one set of insomnia and sleep quality self-assessment questions 71 at a time, and performs a next set of insomnia and sleep quality self-assessment questions 71 after the corresponding self-assessment items 711 have been selected, but this is not a limitation. In other embodiments, multiple sets of insomnia and sleep quality self-assessment questions 71 may be displayed at the same time. The sources of the insomnia and sleep quality self-assessment questions 71 may be from the existing evaluation table as shown in FIGS. 8 and 9, but this is not a limitation.

When the control unit 6 executes the daytime relaxation-assisted program at the current time from 8:00 a.m. to 5:00 p.m., the control unit 6 controls the virtual image to display a natural scene including a mountain, a sea or a lake during the daytime, and plays sound effects with the earphones 21 so that the user is immersed in the experience of the natural scene. For example, when the user selects a beach, the virtual image displays a sea and a wave, and the sound effects broadcast a wave sound. A corresponding change may occur as the virtual reality unit 2 changes directions.

When the control unit 6 executes the nighttime anti-insomnia-assisted program at the current time from 7:00 p.m. to 12:00 p.m., the control unit 6 controls the virtual image to display a scene for relaxing before sleeping, such as a starry sky at night, and to play sound effects with the earphones 21 so that the user is immersed in the experience of a natural nighttime scene. Sound effects with a low volume are played with the earphones 21, so that the user experiences a virtual nature nighttime.

The user may perform the evaluation program before use to obtain a pre-operation insomnia and sleep quality result score, and then perform a evaluation program after the next operation of sleep to obtain a new insomnia and sleep quality result score. Thus, the improvement degree of insomnia can be seen from the two insomnia and sleep quality result scores, which can be used as a record and as a driving force for further use of the user.

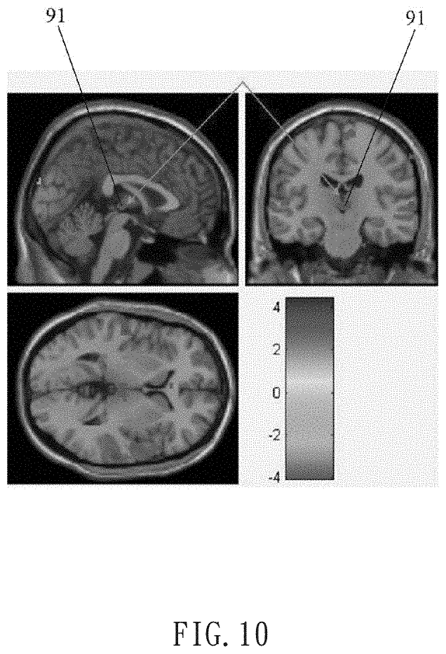

With reference to FIG. 10, the applicant performs an experiment with the training device for improving insomnia, and performs the nighttime anti-insomnia-assisted program with eight subjects. The first predetermined time is 30 minutes, the frequency of the second main carrier frequency tone is 300 Hz, the second high binaural frequency difference is 10 Hz, and the second low binaural frequency difference is 2 Hz. The program of decreasing the first binaural frequency difference by 1 Hz after each interval of 1-2 minutes gradually decreases the second binaural frequency difference from 10 Hz to 2 Hz in sequence. After the end, magnetic resonance imaging (MRI) images are taken for one of the subjects. It can be seen that activation occurs at the pineal body of the MRI brain (i.e. the red activated region 91 indicated by the blue arrow in FIG. 10). Thus, it is sufficient to prove that after performing the nighttime anti-insomnia-assisted program, the nighttime binaural beats with frequency following response can activate the pineal body of the human brain, which is the main organ of the human body for secreting melatonin. The autocrine melatonin can improve the symptoms of insomnia.

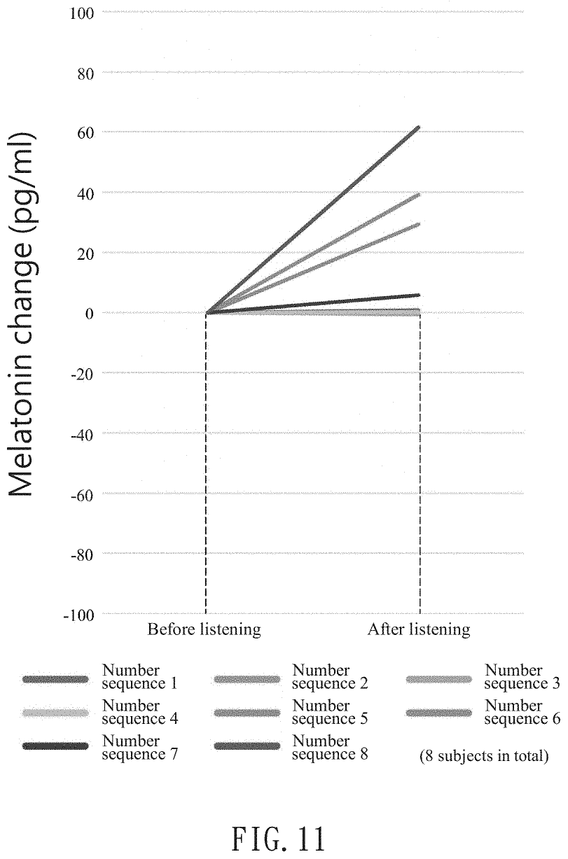

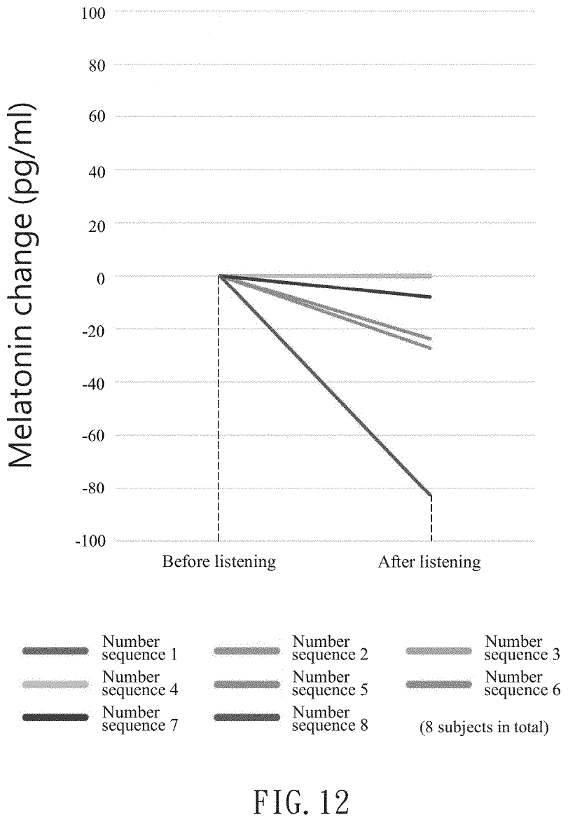

Referring to FIGS. 11 and 12, the applicant further measures the changes in melatonin of the subjects after the nighttime anti-insomnia-assisted program and after listening to general music. As shown in FIG. 11, it can be seen that most of the subjects have a tendency to increase melatonin after receiving the nighttime binaural beats with frequency following response. However, as shown in FIG. 12, the subject who does not receive the nighttime binaural beats with frequency following response but only listens to general music has a slight tendency to decrease melatonin, indicating that listening to the nighttime binaural beats with frequency following response can effectively increase melatonin and thus improve insomnia

Referring to FIGS. 1, 6 and 13, the user can further lie on the vibration bed 5. The insomnia can be improved by applying a vibration frequency of between 6-53 Hz to the user via the vibration bed 5.

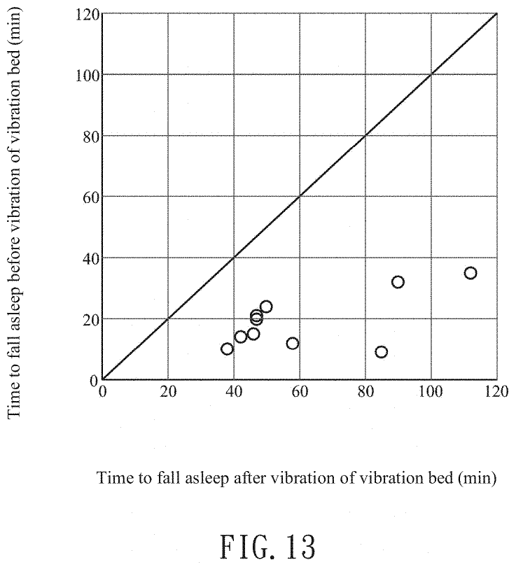

The applicant conducts the test on ten subjects, and obtains the test results as shown in FIG. 13 after the polysomnography (PSG) examination and standard brain wave interpretation. It can be seen that the time of falling asleep during the lying vibration bed of all subjects is shortened. Taking the rightmost subject as an example, it takes 113 minutes (see the horizontal axis) to fall asleep before the vibration bed vibrates, while it takes only 35 minutes (see vertical axis) to fall asleep after the vibration bed vibrates. After the paired t test, the p value is statistically 0.000122. It is significant to show the statistical value, which indicates that the insomnia condition can be effectively improved after vibrating the bed.

In addition, after physical stimulation with the electrical stimulation agents 31, the deep sleep stage N3 is increased by 10 minutes on average in six patients with insomnia, which shows significant improvement of sleep quality. The result scores of insomnia and sleep quality obtained by the evaluation program after physical stimulation on the acupoint agents 41 in six patients with insomnia show that the sleep quality, sleep time and sleep efficiency are significantly improved.

In summary, by providing the virtual reality unit 2 and the control unit 6, the control unit 6 controls the earphones 21 to play the daytime binaural beats with frequency following response and the nighttime binaural beats with frequency following response when the daytime relaxation-assisted program and the nighttime anti-insomnia-assisted program are executed, respectively. Also, in cooperation with the electrical stimulation agents 31, the acupoint agents 41 and the vibration bed 5, the user can achieve the effect of improving insomnia Thus, the object of the present invention can be achieved.

It should be understood that the above-mentioned is only embodiments of the invention, which should not limit the scope of the invention. Simple equivalent changes and modifications made without departing from the scope of the invention and according to the content of the description are covered by the scope of the invention.

What is claimed is:

1. A training device for improving insomnia, comprising:
a virtual reality unit configured for being worn by the head of a user, wherein the virtual reality device comprises two earphones, and an eye cover connected to front sides of the earphones and used for displaying a virtual image; and
a control unit configured for being electrically connected to the virtual reality unit, with an electronic clock capable of indicating a current time built in, and pre-storing digital information of a daytime binaural beats with frequency following response and a nighttime binaural beats with frequency following response, wherein when the current time is from 8:00 a.m. to 5:00 p.m. the control unit can be used for executing a daytime relaxation-assisted program; when the current time is from 7:00 p.m. to 12:00 p.m. the control unit can be used for executing a nighttime anti-insomnia-assisted program; when the control unit executes the daytime relaxation-assisted program, the control unit controls the earphone to play the daytime binaural beats with frequency following response, and stop playing after continuing for a first predetermined time, wherein the daytime binaural beats with frequency following response has a first main carrier frequency tone and a first loading frequency tone which differs from the first main carrier frequency tone by a first difference frequency rate; the first main carrier frequency tone and the first loading frequency tone are respectively played via the earphones simultaneously; the first binaural frequency difference gradually decreases from a first high binaural frequency difference to a first low binaural frequency difference within the first predetermined time; when the control unit executes the nighttime anti-insomnia-assisted program, the control unit controls the earphones to play the nighttime binaural beats with frequency following response, and stops playing after continuing for a second predetermined time; the nighttime binaural beats with frequency following response has a second main carrier frequency tone and a second loading frequency tone which differs from the second main carrier frequency tone by a second binaural frequency difference; the second main carrier frequency tone and the second loading frequency tone are respectively played via the earphones simultaneously; and the second binaural frequency difference gradually decreases from a second high binaural frequency difference which is not higher than the first low frequency to a second low frequency within the second predetermined time.

2. The training device for improving insomnia according to claim 1, wherein the first high binaural frequency difference is 20 Hz; the first low binaural frequency difference is 10 Hz; the second high binaural frequency difference is 10 Hz; and the second low binaural frequency difference is 2 Hz.

3. The training device for improving insomnia according to claim 2, wherein, when the daytime relaxation-assisted program is executed, the control unit controls the eye cover to project light with a wavelength of 465-495 nm and an illumination of 2500-3000 lux via the virtual image; and when the nighttime anti-insomnia-assisted program is executed, the control unit controls the eye cover to project light with a wavelength of 630-670 nm and an illumination of less than 30 lux via the virtual image.

4. The training device for improving insomnia according to claim 2, wherein, if the control unit is requested to perform the daytime relaxation-assisted program when the current time is a time other than 8:00 a.m. to 5:00 p.m., the control unit does not perform the daytime relaxation-assisted program and displays an unusable warning message on the virtual image; and when the current time is a time other than 7:00 p.m. to 12:00 p.m. and if the control unit is requested to perform the nighttime anti-insomnia-assisted program, the control unit does not perform the nighttime anti-insomnia-assisted program and displays the unusable warning message on the virtual image.

5. The training device for improving insomnia according to claim 1, further comprising an electronic stimulation unit, wherein the virtual reality unit further comprises a top-side frame connected between the earphones and upwardly across the top of the user's head, the top-side frame having a top region located at the center between the earphones, two side regions respectively connected between the earphones and the top region, and two connection regions respectively extending forwardly from the side regions;
the electronic stimulation unit comprises a plurality of electrical stimulation agents provided at the connection regions and electrically connected to the control unit; the electrical stimulation agents are used for physical stimulation of one of outputting a current to perform transcranial direct current stimulation and outputting an electromagnetic pulse to perform transcranial magnetic stimulation on the head of the user; and the electrical stimulation agents respectively correspond to an F3 position and an F4 position in international 10-20 system brain wave electrode positions.

6. The training device for improving insomnia according to claim 5, wherein when the electrical stimulation agents is in the form of outputting direct current, the current magnitude is 0.5 mA-2 mA, and the current density is 0.03 mA/cm2-0.09 mA/cm2; and when the electrical stimulation agents is in the form of outputting electromagnetic pulse, the electromagnetic frequency is 1-20 Hz.

7. The training device for improving insomnia according to claim 1, further comprising an acupoint stimulation unit, wherein the virtual reality unit further comprises a top side frame connected between the earphones and upwardly across the top of the user's head, the top side frame having a top region located at the center between the earphones, two side regions respectively connected between the earphones and the top region, a front extension region extending forwardly from the top region, and a rear extension region extending rearwardly from the top region; the acupoint stimulation unit comprises a plurality of acupoint agents electrically connected to the control unit, the acupoint agents being used for outputting physical stimulation of laser light to the user, one of the acupoint agents is adjustably disposed at the center of the top region to correspond to the Baihui acupoint of the user; and four of the acupoint agents are adjustably disposed at the side regions, the front extension region, the rear extension region, and a position at a distance from the center of the top region by a finger distance to correspond to four Sishencong acupoints of the user, respectively, the finger distance being substantially two to three centimeters.

8. The training device for improving insomnia according to claim 1, further comprising a vibration bed electrically connected to the control unit and controllable to generate vibration, the vibration bed having an adjustable vibration frequency of 6-53 Hz.

9. The training device for improving insomnia according to claim 1, wherein the control unit is further configured for executing a audio-visual education program; and when the control unit executes the audio-visual education program, the control unit controls the virtual image to display a teaching course, and generates a teaching image and sound for teaching insomnia improvement in cooperation with the virtual reality unit and earphones.

10. The training device for improving insomnia according to claim 1, wherein the virtual reality unit further comprises an operation button electrically connected to the control unit; the control unit is further configured for executing an evaluation program; when the control unit executes the evaluation program, the control unit controls the virtual image to sequentially display a plurality of insomnia and sleep quality self-assessment questions for assessing insomnia conditions, each insomnia and sleep quality self-assessment question having a plurality of self-assessment items, and the selected self-assessment items can be changed by changing the direction of the eye cover; an insomnia and sleep quality self-assessment score corresponding to the selected self-assessment item is generated after confirmation via the operation button for storage;
and after all the insomnia and sleep quality self-assessment items have been answered, all the insomnia and sleep quality self-assessment scores are summed up to generate an insomnia and sleep quality result score for storage.
